(19) United States
(12) Patent Application Publication
(10) Pub. No.: US 2026/0168119 A1
(43) Pub. Date: Jun. 18, 2026

(54) Electrolyzer Loop Augmentation for Chemical Production Line with Carbon Monoxide Tail Gas

(71) Applicant: Dioxycle, Saint Ouen sur Seine (FR)

(72) Inventors: Sarah Lamaison, Paris (FR); Paul S Wallace, Paris (FR); Jonathan Maistrello, La Garenne Colombes (FR)

(21) Appl. No.: 19/414,379

(22) Filed: Dec. 10, 2025

Related U.S. Application Data
(60) Provisional application No. 63/733,429, filed on Dec. 12, 2024.
(60) Provisional application No. 63/734,033, filed on Dec. 14, 2024.

(51) Int. Cl. C25B 15/08; B01D 53/04; B01D 53/14; B01J 8/04; C01B 3/48; C07C 29/152; C25B 1/02; C25B 3/03; C25B 3/07; C25B 3/25

(52) U.S. Cl. CPC C25B 15/087; B01D 53/0462; B01D 53/1475; B01J 8/0492; C01B 3/48; C07C 29/152; C25B 1/02; C25B 3/03; C25B 3/07; C25B 3/25; C25B 15/083; B01D 2252/2021; B01D 2256/20; B01D 2256/24; B01D 2257/504; B01D 2257/70; B01D 2257/80; C01B 2203/0283

(57) ABSTRACT

This disclosure relates to systems and methods for valorization of carbon monoxide tail gas streams for chemical production lines. A disclosed system includes a chemical production line with a separator to remove a product gas from a tail gas that includes carbon monoxide, separators to remove a second product gas from the tail gas, and electrolyzer systems to convert carbon monoxide in the tail gas to other useful chemicals including the second product gas. The electrolyzer products are recycled back to one or more separation stages.

### CROSS REFERENCE TO RELATED APPLICATIONS

This application claims the benefit of U.S. Provisional Pat. App. No. 63/733,429, filed on Dec. 12, 2024, and U.S. Provisional Pat. App. No. 63/734,033, filed on Dec. 14, 2024, both of which are incorporated by reference herein in their entireties for all purposes.

### BACKGROUND

In order to combat global warming, there is an urgent need to displace virgin-fossil-resource-based fuels and chemicals with low-carbon-intensity fuels and chemicals. Producing chemicals from hydrocarbons without releasing carbon dioxide (CO₂) emissions through strategies such as electrification of heat production to replace traditional gas-fired heaters is an area of technology being investigated. The production of hydrogen, methanol, and other bulk chemicals from fossil-derived feedstocks typically involves reforming, synthesis, and purification steps that generate process off-gases containing carbon monoxide (CO), CO₂, and inert species such as nitrogen and argon. In conventional industrial practice, these tail gas streams are commonly recycled, combusted for low-grade heat, or routed to thermal oxidizers to prevent the accumulation of inerts in the main process loop when portions are recycled. CO₂ can be sequestered or otherwise managed. While these approaches allow the primary chemical product to be manufactured to specification, they routinely result in the loss of valuable carbon and hydrogen content, increased energy consumption, and additional CO₂ emissions.

Some key emerging technologies employ the conversion of CO to other valuable chemicals through electrolysis. These systems are capable of processing CO streams of varying purity and can generate a range of oxygenated and hydrocarbon products, often with co-production of hydrogen or oxygen. However, large-scale deployment of such technology has been limited in part by the availability of suitably priced CO feedstocks. Utilizing CO that is currently inefficiently used for heating or otherwise discarded is beneficial, but thus far process schemes that provide improved pathways for chemical valorization without requiring wholesale redesign of existing production plants are uncommon.

### SUMMARY

This disclosure relates to integrated chemical production systems and methods in which a CO-containing tail gas from a chemical production line is routed to a CO electrolyzer, rather than being combusted, vented, or recycled in a manner that results in loss of chemical value. The electrolyzer can convert at least a portion of the CO into valuable products such as olefins or oxygenates. A separation system produces a CO-rich stream for electrolysis, while recovering additional streams including hydrogen and methane for recycling to other portions of the system. The disclosed systems may be applied as retrofits to existing production lines, such as blue/gray hydrogen or methanol plants, or incorporated into new builds, enabling the valorization of tail gases and reduction of carbon emissions. In conventional chemical production lines, CO in a tail gas is often present in low amounts alongside other constituents that make it economically difficult to separate the CO into a high value CO stream. For similar reasons, high methane, nitrogen, and argon content (as well as low hydrogen content) in the tail gas stream make other valorization pathways such as conversion to methanol impractical, even if additional hydrogen is added.

In specific embodiments, a lower-purity CO-containing stream is suitable for feeding a CO electrolyzer. The tail gas can be separated using, for example, a cryogenic separation system. Furthermore, the electrolyzer product stream can also be recycled back into the cryogenic separation systems. This allows the same separator to be used both to purify the tail gas feed to a level suitable for use as a feed to a carbon monoxide electrolyzer and purify a secondary product gas (e.g., ethylene) at the same time. Other components such as hydrogen and methane can be recycled to other sections of the chemical production line or the separator. Electrolyzers can also produce highly concentrated oxygen streams that can be directly fed to elements of the chemical production line, such as a partial oxidation reactor or thermal oxidizers. The systems and methods described herein provide multiple pathways for improving carbon utilization and overall plant efficiency by converting a low-value or negatively valued gas stream into one or more high-value chemical products.

In specific embodiments, a chemical production line is augmented with a CO-electrolyzer subsystem and an integrated multi-stage cryogenic separator such that the same cryogenic system simultaneously purifies CO-containing tail gas from the chemical production line and gaseous products from the CO electrolyzer. This dual-use configuration enables extraction of olefins, oxygenates, noble gases, hydrogen, methane, and nitrogen purge streams with minimal carbon loss.

In specific embodiments of the invention, a system is provided. The system includes a chemical production line with an output comprising a first product gas and carbon monoxide, a first separator configured to separate the output into a volume of first product gas and a tail gas comprising a volume of carbon monoxide, and a second separator configured to separate a volume of a second product gas from the tail gas. The system also includes a carbon monoxide electrolyzer configured to receive at least a portion of the tail gas and convert at least a portion of the volume of carbon monoxide contained therein into a second volume of the second product gas and a recycle conduit configured to return gaseous products of the carbon monoxide electrolyzer to the second separator.

In specific embodiments of the invention, a method is provided. The method includes producing a first product gas and a tail gas containing a volume of carbon monoxide, separating a first volume of the first product gas from the tail gas containing the volume of carbon monoxide in a first separator, and separating a first volume of a second product gas from the tail gas in a second separator. The method further includes electrolyzing, in a carbon monoxide electrolyzer, at least a portion of the tail gas to convert at least a portion of the volume of carbon monoxide into an electrolyzer product gas having a second volume of the second product gas and recycling at least a portion of the electrolyzer product gas to the second separator.

In specific embodiments of the invention, a system for producing blue hydrogen is provided. The system includes a reforming reactor, selected from an autothermal reformer, a partial-oxidation reactor, and a steam-methane reformer, that produces a reforming reactor output comprising a first volume of carbon monoxide and a first volume of hydrogen, a catalytic water-gas-shift reactor configured to produce, from the reforming reactor output, a shifted output stream comprising a second volume of carbon monoxide, a second volume of hydrogen, and a volume of carbon dioxide, and a first separator configured to separate the shifted output stream into a volume of hydrogen and a tail gas stream comprising the second volume of carbon monoxide, the first separator selected from a pressure swing adsorber, a membrane separator, and a cryogenic separator. The system also includes a carbon-monoxide electrolyzer configured to receive at least a portion of the tail gas stream and to produce an electrolyzer product gas stream comprising a second product gas from at least a portion of the second volume of carbon monoxide and a second separator comprising a cryogenic separation system, the cryogenic separation system configured to separate each of the tail gas stream and the electrolyzer product gas stream into one or more of (i) a hydrogen stream, (ii) a carbon-monoxide stream, (iii) a nitrogen-rich purge stream, and (iv) a stream containing a volume of the second product gas.

In specific embodiments of the invention, a system for producing methanol is provided. The system includes a reforming reactor, selected from an autothermal reformer, a partial-oxidation reactor, and a steam-methane reformer, that receives a hydrocarbon as an input and produces a reforming reactor output comprising carbon monoxide, carbon dioxide, and hydrogen, a methanol synthesis reactor configured to produce, from the reforming reactor output, an output gas stream comprising methanol and residual carbon monoxide, and a first separator configured to separate the output gas stream into a volume of methanol and a tail gas stream comprising a volume of the residual carbon monoxide, the first separator selected from a pressure swing adsorber, a membrane separator, and a cryogenic separator. The system also includes a carbon monoxide electrolyzer configured to receive at least a portion of the tail gas stream and to produce an electrolyzer product gas stream comprising a second product gas from at least a portion of the volume of the residual carbon monoxide and a second separator comprising a cryogenic separation system, the cryogenic separation system configured to separate each of the tail gas stream and the electrolyzer product gas stream into one or more of (i) a hydrogen stream, (ii) a carbon-monoxide stream, (iii) a nitrogen-rich purge stream, and (iv) a stream containing a volume of the second product gas.

### BRIEF DESCRIPTION OF THE DRAWINGS

The accompanying drawings illustrate various embodiments of systems, methods, and various other aspects of the disclosure. A person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 provides a diagram of a chemical production line in accordance with specific embodiments of the inventions disclosed herein.

FIG. 2 provides a diagram of a natural gas reforming chemical production line in accordance with specific embodiments of the inventions disclosed herein.

FIG. 3 provides a diagram of a blue hydrogen production line with a partial oxidation (POx) reactor combined with separators and electrolyzers in accordance with specific embodiments of the inventions disclosed herein.

FIG. 4 provides a diagram of a blue hydrogen production line with an autothermal reformer (ATR) reactor combined with separators and electrolyzers in accordance with specific embodiments of the inventions disclosed herein.

FIG. 5 provides a diagram of a detailed view of separators and electrolyzer systems in accordance with specific embodiments of the inventions disclosed herein.

FIG. 6 provides a diagram of a methanol production line combined with separators and electrolyzers in accordance with specific embodiments of the inventions disclosed herein.

FIG. 7 provides a diagram of a detailed view of an alternate configuration of separators and electrolyzer systems in accordance with specific embodiments of the inventions disclosed herein.

FIG. 8 provides a diagram of a process of operating a chemical production line in accordance with specific embodiments of the inventions disclosed herein.

### DETAILED DESCRIPTION

Reference will now be made in detail to implementations and embodiments of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Different systems and methods for augmenting chemical lines for CO electrolysis are described in detail in this disclosure. The methods and systems disclosed in this section are nonlimiting embodiments of the invention, are provided for explanatory purposes only, and should not be used to constrict the full scope of the invention. It is to be understood that the disclosed embodiments may or may not overlap with each other. Thus, part of one embodiment, or specific embodiments thereof, may or may not fall within the ambit of another, or specific embodiments thereof, and vice versa. Different embodiments from different aspects may be combined or practiced separately. Many different combinations and sub-combinations of the representative embodiments shown within the broad framework of this invention, that may be apparent to those skilled in the art but not explicitly shown or described, should not be construed as precluded.

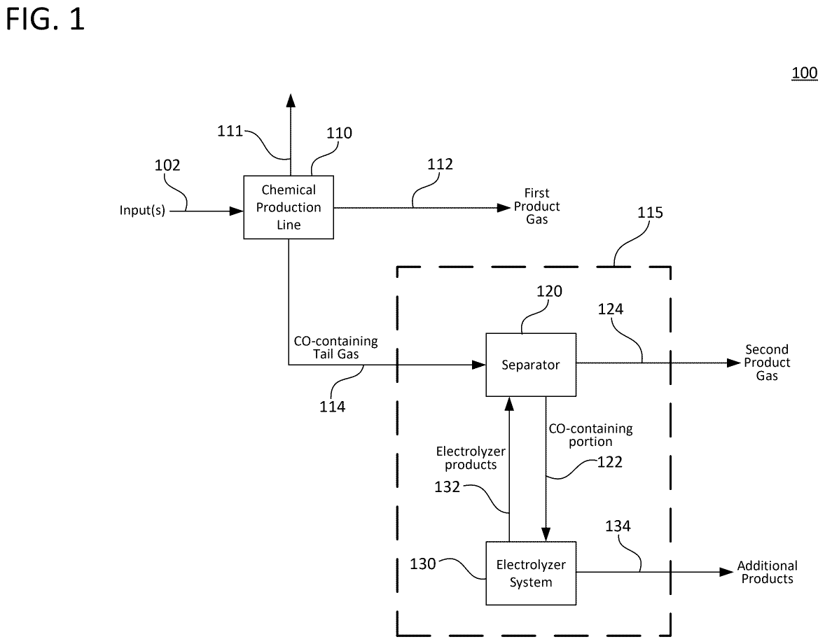

FIG. 1 shows a simplified diagram of an integrated production system 100 in which a conventional chemical production line is modified to incorporate a downstream subsystem 115 including a CO-electrolyzer according to specific embodiments of the invention. The electrolyzer can be a zero-gap, membrane-electrode-assembly (MEA), or alkaline flow-cell electrolyzer. The anode may oxidize water, hydrogen, or another oxidation substrate. The cathode catalyst may be copper based, silver based, or a bimetallic catalyst suitable for CO reduction. System 100 incorporates separation stages and electrolysis to utilize byproducts that would have been otherwise discarded or inefficiently burned for heating. The conventional chemical production line may exist already so that the CO-electrolyzer subsystem 115 is retrofitted into the system, or a new system can be designed from the beginning to incorporate subsystem 115. System 100 includes one or more feed inputs 102 introduced into a chemical production line 110 which produces various products including a first product gas stream 112 suitable for commercialization and a tail-gas stream 114 comprising CO and possibly other components depending on the method of separation of the tail gas stream 114 from the first product gas stream 112. In many conventional systems, this tail gas stream is combusted for low-value heat recovery for steam generation, for heating of feed inputs, and the like, or is discarded to purge inert gases or other unwanted constituents. In the configuration shown in FIG. 1, the tail gas stream 114 is instead diverted to a downstream processing subsystem 115. The chemical production line 110 may also produce by-product streams 111, which may include steam when water is used for cooling components. Internally produced steam can be used externally, or internally within the production line 110 or subsystem 115 for various processes.

In specific embodiments, the tail gas stream 114 is fed to a separator 120 that separates its input stream into a second product gas stream 124 and a CO-containing stream 122. The CO-containing stream 122 is supplied to an electrolyzer system 130 configured to electrochemically convert at least a portion of the carbon monoxide to oxygenates, olefins, or other upgraded products using an oxidation substrate such as water or hydrogen. The electrolyzer system 130 produces one or more electrolyzer product streams 132 that are returned to the separator 120 for further purification. In this case, the separator acts as a combined separator for both the tail gas stream 114 and the electrolyzer product stream 132. The electrolyzer system 130 may also produce one or more additional product streams 134 that are withdrawn from the system 100 separately. Additional products can be liquid products directly output or can be routed to other subsystems within subsystem 115 for purification or other processing. In specific embodiments, the second product gas stream 124 comprises second product gas already present in tail gas stream 114. In specific embodiments, the one or more electrolyzer product streams 132 include a volume of the second product gas converted from CO. When the electrolyzer product stream 132 is recycled to the separator, the second product gas stream 124 thus comprises second product gas created in the electrolyzer system 130. In specific embodiments, subsystem 115 can be configured so that the tail gas stream 114 is first fed into the electrolyzer system 130, and the output of the electrolyzer system 130 passes to the separator 120 for purification and recycling.

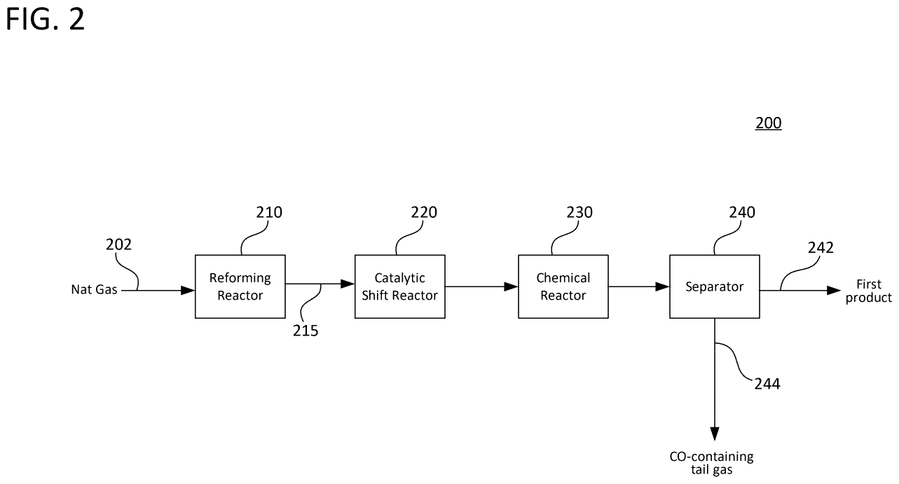

FIG. 2 shows a diagram of a natural gas reforming chemical production line according to specific embodiments of the invention. Natural gas is commonly used as a feedstock for producing various chemicals. Chemical line 200 is one example of a chemical production line 110 as shown in FIG. 1. Natural gas (or methane) stream 202 is input to a reforming reactor 210. In specific embodiments, the reforming reactor 210 can be a partial oxidation (POx) reactor, an autothermal reformer (ATR), or a steam methane reformer (SMR). The output 215 of the reforming reactor 210 comprises a syngas with a hydrogen/CO ratio dependent on the type of reforming reactor as well as chosen reactor conditions. Syngas output 215 can pass first to a catalytic shift reactor 220, and then to a chemical reactor 230. A separator 240 can separate a first product 242 from a CO-containing tail gas 244. Reactions in the reforming reactor and the catalytic shift reactor can include the POx reaction, the SMR reaction, and the water gas shift (WGS) reaction as follows in Eqs. 1-3:

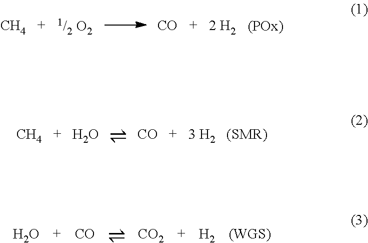

Though the above reactions include methane as the reactant, other hydrocarbons present in natural gas will react similarly in a known manner. Depending on the desired output of the chemical line 200, a catalytic shift reactor 220 can be used to adjust the syngas ratio using the WGS reaction. In specific embodiments, the reforming reactor 210 and the catalytic shift reactor 220 can be combined in the same unit. In specific embodiments, the output of the reforming reactor 210 can already have a syngas ratio composition within a desirable range for the chemical reactor, and thus the catalytic shift reactor 220 can be omitted from the process flow. For example, in a chemical line producing methanol, a methanol reactor can be configured to accept one or more of CO and CO₂ along with hydrogen as feeds. As long as these levels are within acceptable ranges, the output of the reforming reactor can be used directly in a methanol reactor without modification. In specific embodiments, the catalytic shift reactor 220 already produces the desired primary output of the chemical production line, in which case the downstream chemical reactor 230 can be omitted. One example of this is when hydrogen is the primary output, where a catalytic shift reactor can undergo the WGS to a desired level of completion that converts additional CO into hydrogen.

Although hydrogen and methanol production lines are used as examples in this disclosure, many other chemical processes provide a separated tail gas containing CO that could be used as feed to separators and electrolyzer systems as disclosed herein. Some non-limiting examples where the input can be a syngas derived from a reforming reactor include ammonia and ammonia derivatives, the Fischer-Tropsch process, dimethyl ether (DME) and other oxygenates, alcohol production, acetic acid and other carboxylates, direct reduced iron (DRI) steelmaking, and the like.

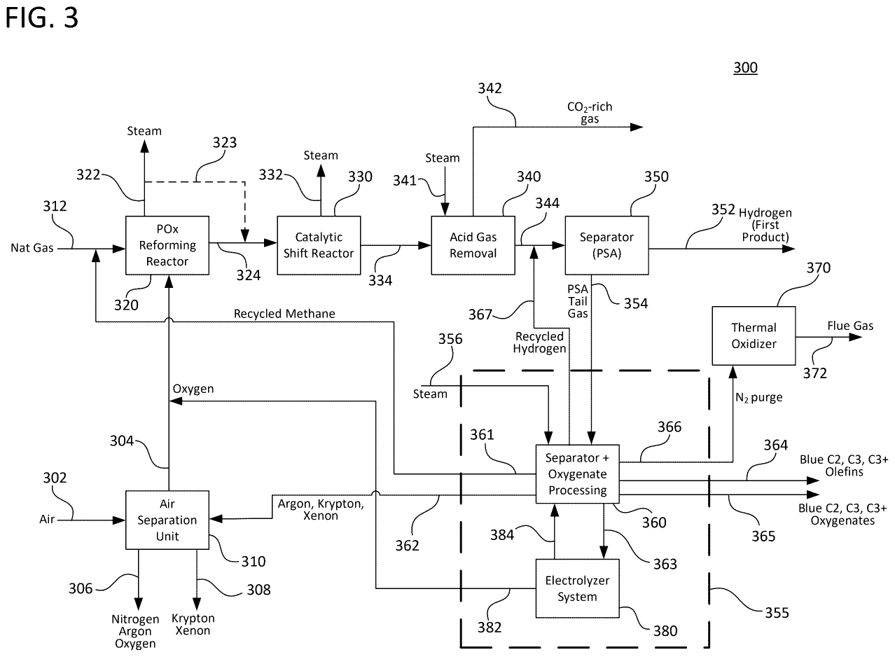

FIG. 3 illustrates a diagram of a blue hydrogen production system 300 according to specific embodiments of the invention. System 300 is a specific example of system 100, where the chemical production line 110 is a natural gas reforming chemical production line such as chemical line 200. System 300 is configured to produce blue hydrogen from natural gas or methane using a POx reactor as the reforming reactor. In specific embodiments, the chemical production line comprises an air separation unit (ASU) 310, a POx reactor 320, a catalytic shift reactor 330, an acid-gas removal unit 340, and a separator 350. A subsystem 355 comprising separation stages and an electrolyzer system corresponds to subsystem 115 of FIG. 1. In the illustrated configuration, natural gas 312 enters a POx reactor 320 along with an oxygen stream 304 delivered from an ASU 310. An ASU 310 with incoming feed air stream 302 is commonly used to provide substantially pure (>99 mol %) oxygen for the POx reactor 320. Although most of the noble gases are removed from the oxygen stream 304, the oxygen stream 304 may still contain commercially useful amounts of argon, krypton, and xenon. In current blue hydrogen plants, the argon, krypton, and xenon are concentrated in a separator tail gas volume and are lost with the nitrogen purge to a thermal oxidizer or if the tail gas is combusted in a fired heater for heating other input feeds. The POx reactor 320 outputs a syngas stream 324 comprising CO and hydrogen, though the ratios may vary depending on the configuration of the reactor. The syngas output may also include various amounts of CO₂, methane, steam, and inert gases.

Cooling within the reactor 320 can also produce a high-pressure steam stream 322. In specific embodiments, a portion of the steam stream 323 can be added to the downstream input to the next reactor to facilitate the WGS reaction. In specific embodiments, the syngas stream 324 is then directed to a catalytic shift reactor 330, where a portion of the CO reacts with steam using the WGS reaction to increase the hydrogen yield. An excess steam stream 332 may optionally be produced here as well. In conventional blue hydrogen production lines, remaining CO can be used as a low value, high-carbon heat source when present in the tail gas. In these cases, conditions are often chosen with excess steam and temperatures to maximize the conversion of CO and water into hydrogen and CO₂, though this may use more of the valuable high-pressure steam generated by the reforming reactor than would be otherwise desired. In specific embodiments, these conditions can still be used if the goal is to maximize the hydrogen output of the system overall, i.e., to drive the reaction from CO to hydrogen as far as possible. Alternatively, in system 300, as CO can be valorized into higher value chemicals, the ratio of CO/hydrogen can be increased if desired. This produces less net CO₂ as well as using less steam and can allow selection of a catalytic shift reactor 330 that is smaller and uses less heating/cooling capacity as compared to conventional designs. Moreover, since the equilibrium of the WGS reaction favors hydrogen generation at lower temperatures, a longer catalyst bed designed for use at lower temperatures may be needed to drive the reaction toward more hydrogen production. When a lower hydrogen:CO ratio is acceptable as part of gas stream 334, then a high temperature shift reactor can be used that is either smaller or uses fewer stages for conversion. In specific embodiments, the catalytic shift reactor 330 can be omitted from the system if the syngas stream 324 alone is acceptable.

The gas stream 334 is delivered to an acid-gas removal unit 340 that separates a CO₂-rich stream 342 from a treated gas stream 344. The acid-gas removal unit 340 can optionally use a steam input 341 for heating. The CO₂-rich stream 342 can be routed for CO₂ sequestration or otherwise utilized. The treated gas stream 344 flows to a gas separator, for example, a pressure swing adsorption (PSA) separator 350, which produces a hydrogen-rich product gas stream 352 and a tail gas stream 354 containing methane, hydrogen, CO₂, CO, and other inert components such as nitrogen. Tail gas stream 354 is directed to a separator/electrolyzer subsystem 355, shown within the dashed boundary. First, the tail gas is fed into a separator and oxygenate processing subsystem 360. In specific embodiments, the separator and oxygenate processing subsystem 360 can include one or more cryogenic separator stages that can separate out various gaseous products. Gaseous products can include a C2, C3, and/or C3+ olefin stream 364, for example, ethylene. Olefins produced are dependent on the electrolyzer configuration. Gaseous products can include a recycled hydrogen stream 367. Hydrogen stream 367 comprises any hydrogen that was included in tail gas stream 354 as well as hydrogen that may be produced downstream in the electrolyzer 380. The hydrogen stream 367 can be output on its own as a >95 mol % pure hydrogen product, or can be recycled as an input to the separator 350 for further purification. Gaseous products can include a methane stream 361, which can be recycled back to the POx reactor 320. Gaseous products can also include a nitrogen-rich purge stream 366 that can be directed to a thermal oxidizer 370 which outputs a flue stream 372. The nitrogen-purge stream 366 can contain 80 mol % nitrogen or more. This removes nitrogen from the recycle loop to prevent buildup with minimal loss of valuable carbon and hydrogen content. Gaseous products can also include one or more noble gas streams 362 that can be sent back to the ASU 310 or else can be output separately. In specific embodiments, the separator section can include other separation steps to cool, dry, and remove residual CO₂ from stream 354 before further separation. The separator and oxygenate processing subsystem 360 can also recover valuable co-products such as methanol or other light oxygenates, depending on the incoming composition, and output them as a C2, C3, and/or C3+ oxygenate stream 365. Optionally, a steam input 356 can feed the separator and oxygenate processing subsystem 360 for separation or other reactions.

A CO-enriched stream 363 can be supplied to an electrolyzer 380 configured to electrochemically convert CO to higher-value species. Optionally, separator and oxygenate processing subsystem 360 can supply a water stream to the electrolyzer 380. The electrolyzer produces various products that can be separated or processed by separator and oxygenate processing subsystem 360. In specific embodiments, the electrolyzer can produce a high-purity oxygen stream 382 that can be combined with oxygen stream 304 and routed back to the reforming reactor 320. Oxygen produced in an electrolyzer is typically purer than that obtained by an ASU 310, containing almost no nitrogen or noble gas content. This would thereby reduce reliance on the ASU 310 for oxygen and reduce the introduction of inert impurities into the process chain. The electrolyzer product stream 384 is sent back to the separator and oxygenate processing subsystem 360, where olefins and oxygenates can be separately output as previously outlined.

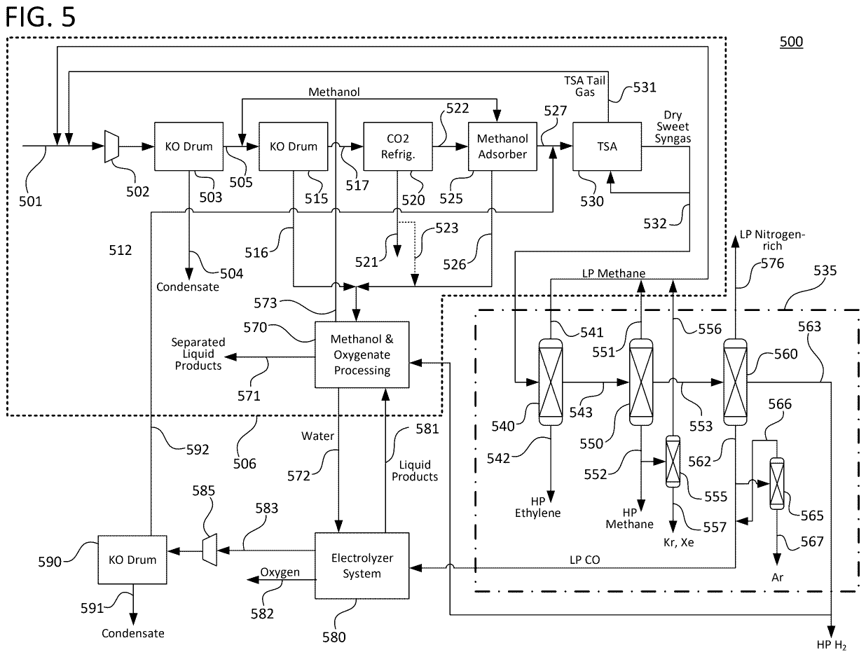

The ASU 310 supplies both oxygen to the reforming reactor 320 and one or more coproduct streams 306 containing nitrogen, argon, or smaller amounts of oxygen, which may be relatively easy to separate in conventional ASU systems. Trace noble gases 308 such as krypton and xenon, can be recovered for additional value, but it is often economically unfeasible to recover them unless the ASU system and volumes of gas are large. By integrating cryogenic separation, methane recycle, and a CO-conversion electrolyzer into the tail gas stream processing pathway, noble gases remain in the system and can be concentrated as the output stream is recycled, which allows economical extraction of the noble gases. More details of this will be presented with respect to FIG. 5.

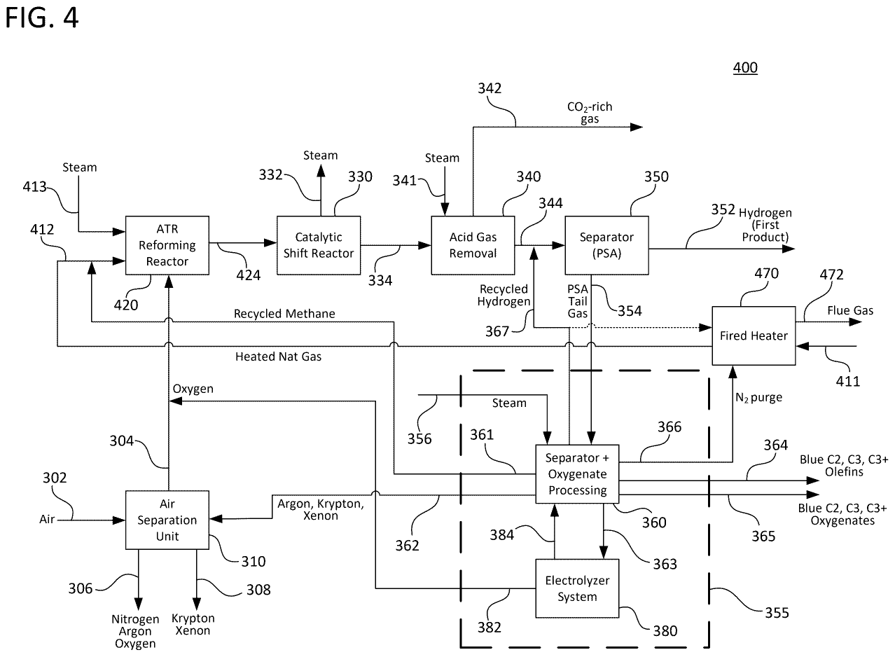

FIG. 4 illustrates a diagram of a blue hydrogen production system 400 according to specific embodiments of the invention. System 400 is very similar to system 300 in that the output is a purified stream of hydrogen, but an ATR reactor 420 is used instead of a POx reactor 320. Most elements of system 400 are the same as in system 300 and will not be discussed separately. When using a POx reactor, partial oxidation of methane and other hydrocarbons is quite exothermic and does not require much preheating of gases, if any. In an ATR, though there is still a POx section of the reactor, it may be beneficial to include a fired heater to pre-heat incoming natural gas feeds for more effective conversion in the SMR portion of the reactor. A fired heater 470 is used to heat incoming natural gas stream 411. The heated natural gas stream 412 is fed as input to the ATR reactor 420, which outputs a syngas stream 424. A steam flow 413 can be added as a separate feed to the ATR reactor 420. A recycled methane stream from the separator can be mixed with the heated stream 412, but in some embodiments, it can also be routed to the fired heater 470 first. In conventional blue hydrogen systems, the CO-rich tail gas has been used for heating of a fired heater such as heater 470, but the CO-rich tail gas can be replaced with hydrogen-rich fuels instead. In specific embodiments, this can be partly or wholly supplied by a portion of recycled hydrogen stream 367. The fired heater may also replace the need for a separate thermal oxidizer 370, since the fired heater can accept the nitrogen-rich purge stream 366 and output a flue gas 472.

In specific embodiments, a blue hydrogen system can be implemented using an SMR reactor instead of a POx or ATR reactor. This configuration is not shown but is substantially similar to system 400 where incoming natural gas stream would need additional heating to drive a favorable equilibrium conversion of hydrocarbon input to a syngas. In this configuration, a separate ASU 310 might not be necessary, as there is no POx reaction that requires a separate oxygen input such as oxygen stream 304.

Both systems 300 and 400 output a CO-containing tail gas stream 354 that can be processed by separator/electrolyzer subsystem 355. FIG. 5 illustrates an integrated separator and electrolyzer subsystem 500 according to specific embodiments of the invention. In this example, subsystem 500 shows a detailed view corresponding to the separator/electrolyzer subsystem 355 in FIGS. 3 and 4. Incoming tail gas stream 501 (which may correspond to tail gas stream 354 in FIGS. 3 and 4) is first routed through several stages to remove residual water and CO₂ from the stream in a first separator section 506 outlined by a dotted line. The tail gas stream 501 first enters a compressor 502 which brings it to high pressure, cooled, and then passed to a knockout drum 503 or a similar gas/liquid separator that removes a condensate 504. Most of the water present is removed from tail gas stream 501 by this step. In specific embodiments, there may be one or more additional inputs to subsystem 500. If pressures, flows, and materials are compatible, chemical streams from different portions of the production line could be mixed into one output such as tail gas stream 501. Certain chemical production lines may have other streams that require their own input line.

Stream 505 is contacted with a portion of a methanol stream 573 to remove any trace moisture. Alternatively, trace moisture can be dried using a liquid drying agent, in a temperature swing adsorber (TSA), and the like. Stream 505 proceeds to a second knockout drum 515, which produces a condensate stream 516 comprising methanol and water, and a dried gas stream 517. In specific embodiments, the methanol/water stream 516 can be sent to a methanol and oxygenate processing unit 570, which can be configured to regenerate or produce methanol. The synthesis reaction is shown as follows in Eq. 4:

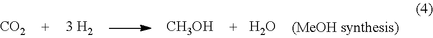

In specific embodiments, methanol and oxygenate processing unit 570 comprises a methanol synthesis reactor, where a volume of CO₂ from the tail gas stream 354 separated out at various points is reacted with a volume of hydrogen. The hydrogen can be a portion of the high-pressure hydrogen stream 563 from the downstream cryogenic system and optionally makeup hydrogen. In some cases, makeup hydrogen can be electrolytically produced from the electrolyzer system. Methanol produced along with hydrogen and unreacted CO₂ can be routed to a separate distillation and separator section for purification. A portion of purified methanol can be recycled for use in the methanol streams 573 used to remove water and CO₂. The methanol and oxygenate processing unit 570 can optionally produce a water stream 572 for use in the electrolyzer system 580.

In specific embodiments, the methanol and oxygenate processing unit 570 can also include components for oxygenate processing. The methanol synthesis reaction can also produce small amounts of ethanol and dimethyl ether. These can be valorized separately. The electrolyzer system 580 can also produce liquid products, for example carboxylates, in a liquid product stream 581. These can also be processed in the oxygenate processing portions into, for example, carboxylic acids like acetic acid. Although unit 570 is depicted is a single system, in specific embodiments, these functions can be separated into separated systems.

A CO₂-refrigeration unit 520 cools dried gas stream 517 to condense almost all the CO₂ contained in stream 517. The condensed CO₂ stream 521 can be sent to sequestration, but alternatively, some or all of stream 521 can be sent as stream 523 to the methanol and oxygenate processing unit 570 for further processing or conversion. The remaining refrigerated gas stream 522 flows to a methanol-based CO₂ adsorber 525, where similarly to earlier at stream 512, a methanol stream is contacted with the refrigerated gas stream 522. This step removes essentially all residual CO₂ from output stream 527 where methanol absorbs the CO₂. A methanol stream 526 with adsorbed CO₂ can also be sent to the methanol and oxygenate processing unit 570. In cases where substantially all the CO₂ in the tail gas is converted to methanol, this would leave the separator portion without significant carbon output. In specific embodiments, downstream electrolyzer product stream 592 can be mixed with stream 527. Stream 527 can be passed to a TSA 530, which removes any remaining moisture, methanol, and oxygenates that would interfere with the downstream cryogenic separators. TSA output stream 532 exits as a dry sweet syngas stream. In specific embodiments, a portion of the TSA output stream 532 can be used as a purge stream to regenerate TSA 530, and the resulting TSA tail gas stream 531 can be passed back to be combined with tail gas stream 354. In specific embodiments, instead of a TSA, a liquid drying agent or other drying unit can be used here to remove residual constituents from stream 527.

In specific embodiments, TSA output stream 532 is routed to a multi-stage cryogenic separation process flow, outlined as section 535. The cryogenic train comprises one or more primary cold separation stages and acts as a combined separator for both the PSA tail gas stream 354 and the electrolyzer product stream 592. Three stages of low-pressure strippers, 540, 550, and 560 are shown here, but additional stages could be used depending on the product mix of the chemical production line and the electrolyzer configuration. The multi-stage cryogenic separation can operate at multiple pressures and temperatures. TSA output stream 532, which in this example includes ethylene produced in the electrolyzer, can be cooled in a multi-stage sequential high-pressure condensing and a low-pressure stripping system. In specific embodiments, a secondary gas product is condensed out of the high-pressure feed gas and stripped in a low-pressure stripper 540, where the product stream is withdrawn as a liquid ethylene bottoms product 542. In specific embodiments, the secondary ethylene product stream can be highly purified (in some cases >99.9 mol % ethylene). Thus, it is important to remove water and other components that may have a lower boiling point than electrolyzer product gases to be separated in the upstream separator section 506. The overhead gas stream 541 from the low-pressure stripper 540 comprises mainly methane and can be recycled back to be combined with tail gas stream 354 for eventual recycling back to the chemical production line. The liquid ethylene bottoms product 542 can be pumped to a high pressure to produce a pure high-pressure ethylene output stream. In specific embodiments, low-pressure stripper 540 can be optimized to increase the purity of the secondary product ethylene while allowing a portion of ethylene into overhead gas stream 541.

Output gas stream 543, still at high pressure, is passed to a second low-pressure stripper 550, which condenses methane and concentrates some noble gases. For clarity, the figure shows the general process flow of TSA output stream 532 through the cryogenic separation stages. Output gas stream 543 is not a physical output of the low-pressure stripper 540, but instead the cryogenic system also includes various refrigeration stages to condense one fraction (e.g., ethylene) and a gas/liquid separator for each stage. The methane-rich bottoms liquid stream 552 is recovered as a recycle stream for upstream reforming. Because it is not intended as a final product, the stripped liquid methane can have a lower purity target than the secondary product ethylene, though the methane-rich bottoms liquid stream 552 is still commonly >80 mol % methane. Methane-rich bottoms liquid stream 552 can be pumped to high pressure and directly returned along stream 361 as an input to the reforming reactor. The methane-rich overhead gas from the second low-pressure stripper 550 is withdrawn as stream 551 and may be combined with overhead gas stream 541 and recycled similarly.

A third low-pressure stripper 560 receives input stream 553 and removes liquid CO as a bottoms liquid 562. The low-pressure stripper 560 produces an overhead nitrogen-rich purge stream 576. Because CO and nitrogen have a similar boiling point, they can be difficult to extract with great purity. In specific embodiments, the low-pressure stripper 560 is configured to withdraw a low-per-pass amount of nitrogen while still producing a nitrogen-rich purge stream 576. The nitrogen-rich purge stream 576 can contain 80 mol % nitrogen or more, which minimizes carbon and hydrogen loss. As a result, the condensed CO-rich bottoms liquid 562 exits the low-pressure stripper 560 at a lower purity (e.g., 50-80 mol % CO). However, a CO electrolyzer can work well with a lower-purity CO feed. The last fraction of TSA output stream 532 is the remaining high pressure hydrogen stream 563. This hydrogen-rich stream has fairly high purity at this stage (e.g. >95 mol % hydrogen). TSA output stream 532 can be pumped to higher pressure and output as additional first product gas when the chemical production line is a blue hydrogen line. If higher purity is desired, the high-pressure hydrogen stream 563 can be sent as recycled stream 367 for additional purification as shown in FIGS. 3 and 4.

In specific embodiments, additional strippers can be added to extract high-value noble gases in the cryogenic separator. In specific embodiments, a portion of the methane-rich bottoms liquid stream 552 can be fed into a small slipstream side stripper 555 to condense a liquid krypton and/or xenon stream 557. This process only extracts a low amount of krypton or xenon per pass, but because the methane-rich bottoms liquid stream 552 is recycled through the overall system, krypton and xenon will eventually return to the cryogenic separator for separation. The small size of the stripper 555 and position within the refrigeration chain allows cost-effective separation of krypton and xenon with minimal additional refrigeration. Overhead gas stream 556 is methane-rich and can be combined with overhead gas stream 541 for recycling. In specific embodiments, a portion of the CO-rich bottoms liquid 562 can be fed into a small slipstream side stripper 565 to condense a liquid argon bottoms product 567. Similar comments about the xenon/krypton stripper apply here as well. One difference is that the overhead stream 566 can be returned to the CO-rich bottoms liquid 562.

CO-rich bottoms liquid 562 is supplied to an electrolyzer system 580. Electrolyzer system 580 converts at least a portion of the CO feed into upgraded products such as ethylene, ethanol, acetic acid, propanol, acrylic acid, glyoxylic acid, or other oxygenates and olefins, depending on catalyst selection. The electrolyzer also produces a high-purity oxygen stream 582 that may be recycled to a reforming reactor to reduce reliance on an ASU. The electrolyzer product gas 583 is recompressed using compressor 585, and the product is separated using a knockout drum 590 into a liquid stream 591 and a gaseous stream 592. The gaseous stream 592 acts as a recycle conduit that can be returned to the cryogenic separation train for recovery of olefinic and oxygenate products alongside components from the original tail gas stream 354.

A carbon monoxide electrolyzer is a device comprising a cathode area where CO reduction takes place, according to equation 5 below, and an anode area where an oxidation reaction takes place on an oxidizing catalyst. The oxidation substrate can be hydroxide, water, dihydrogen gas, halides, organic waste, or any other oxidation substrate. For example, the oxidation can involve water oxidation or hydrogen oxidation according to equations 6 and 7 below respectively.

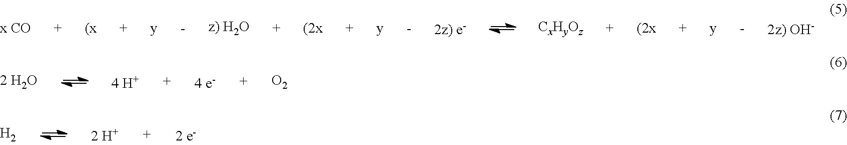

The reactions below can be conducted in accordance with the electrolyzer assemblies described herein. In the diagrams provided herein, only single cells are represented for clarity, but these could be assembled in a plurality of cells, such as in an electrolyzer stack. In the diagrams, a carbon monoxide electrolyzer comprises a cathode comprising a gas-diffusion layer and a cathode catalyst, and the anode comprises an anode catalyst deposited on a transport layer of any shape (such as but not limited to a foam, a mesh, a deposit onto a conductive porous transport layer (PTL), etc.). In this case, the carbon monoxide reduction products include one or more of the following: ethylene (C₂H₄), ethanol (C₂H₅OH), acetic acid (CH₃COOH), propylene (C₃H₆), propanol (C₃H₈O), propionic acid (C₂H₅COOH), oxalic acid (COOH—COOH), acrylic acid (C₂H₃COOH), glyoxylic acid (CHO—COOH). Examples of CO reduction reaction in neutral/alkaline conditions include:

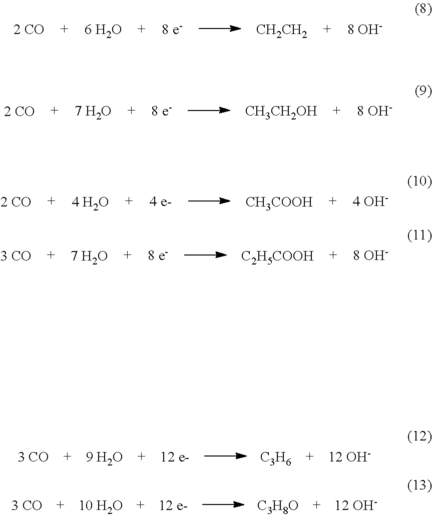

In some embodiments, all the cryogenic product streams are reheated against the mixed feed stream to recover essentially all the refrigeration. An external refrigeration system using either a condensable single mixed refrigerant with a Joule-Thompson expansion valve or a non-condensable refrigerant with an expander is used to provide the net refrigeration required to allow temperature driving forces across the heat exchangers and to provide refrigeration for liquid products pumped to high pressure in the cryogenic separation system.

The configuration as shown in subsystem 500 may be used when the tail gas stream has significant levels of water and/or CO₂ that are removed before other separation. Subsystem 500 also shows cryogenic separation of various gases including a secondary product gas such as ethylene before sending a CO-rich stream to the electrolyzer. However, other configurations are possible depending on tail gas stream 501 composition or other system constraints. In specific embodiments, the tail gas stream can be configured to first pass through the electrolyzer system 580 and then pass to separator sections 506 and 535. In specific embodiments, water and/or CO₂ can be removed in separator section 506, the stream (e.g., the dry, sweet syngas stream 532) can pass to an electrolyzer system 580, and then the electrolyzer output could pass through a cryogenic separator section 535.

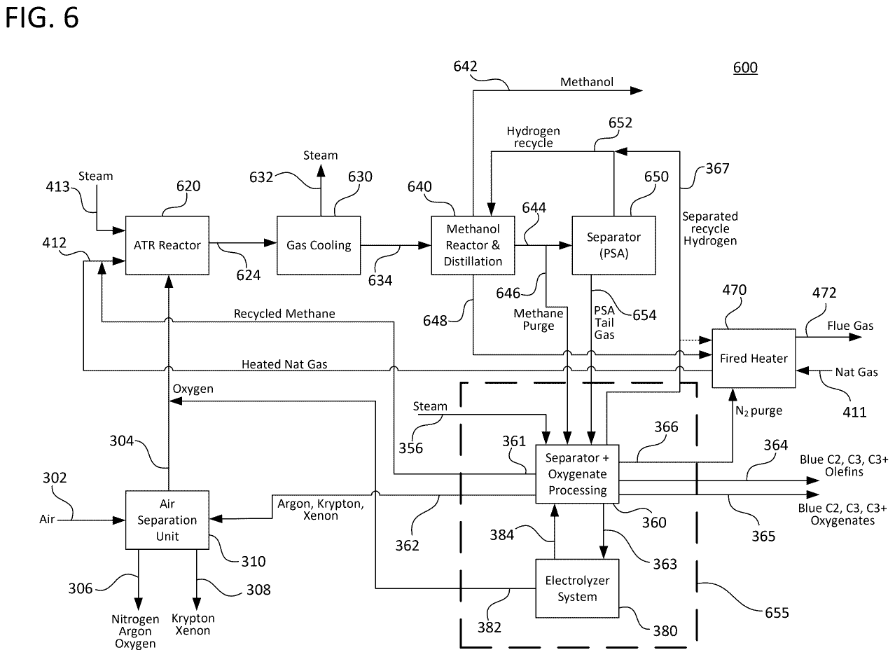

FIG. 6 illustrates a diagram of a methanol production system 600 according to specific embodiments of the invention. System 600 is another specific example of system 100, where the chemical production line 110 is a natural gas reforming chemical production line such as chemical line 200. System 600 is configured to produce methanol from a hydrocarbon source such as natural gas. The methanol producing system 600 uses an ATR as the reforming reactor in the specified configuration. Other configurations can use a POx or an SMR reactor which have been previously described with respect to the blue hydrogen production line. Regardless of the reforming reactor chosen, the output of the reforming reactor is a syngas that includes some amount of CO₂ as well.

In specific embodiments, the chemical production line of system 600 comprises an ASU 310, a reforming reactor 620, a gas cooling unit 630, a methanol reactor and distillation unit 640, and a separator 650. A subsystem 655 comprising separation stages and an electrolyzer system corresponds to subsystem 115 of FIG. 1. Other elements in system 600 that are identical to those in system 400 will have the same figure numbering and will behave substantially similar to elements described previously herein. In the illustrated configuration, heated natural gas stream 412 enters a reforming reactor 620, here configured as an ATR reactor, along with an oxygen stream 304 delivered from an ASU 310. Natural gas is converted in the ATR reactor 620 to a syngas comprising CO and hydrogen, as well as various amounts of CO₂, methane, steam, and inert gases. Output stream 624 is cooled in a gas cooling unit 630. The gas cooling unit may produce high and/or medium pressure steam streams 632.

Cooled gas stream 634 passes to a methanol reactor and distillation unit 640. Unlike the output of the reforming reactors in the hydrogen production systems 300 or 400, there is typically no need for a water gas shift reactor to change the syngas ratios. Methanol reactors can be configured to react hydrogen with one or both of CO and CO₂ input feeds. Methanol produced in the methanol reactor and distillation unit 640 is purified using distillation or other methods and output as stream 642. In specific embodiments, an offgas or byproduct purge stream 648 can be used to provide additional heating in the fired heater 470. An output stream 644 of the methanol reactor and distillation unit 640 from a syngas purge is sent to a separator 650, which in specific embodiments can be a PSA separator. The separator 650 outputs a purified hydrogen stream 652, which can be recycled as an additional hydrogen feed to react with incoming CO and/or CO₂. The PSA tail gas 654 is sent to separation subsystem 655. In specific embodiments, a sidestream purge of the purified hydrogen stream 652 can be done to prevent methane buildup in the loop. Methane acts as an inert gas in the methanol reactor and distillation unit 640, and its presence reduces the possible yield of methanol output. By purging this periodically or continuously, methane buildup can be avoided. In conventional methanol synthesis systems, this sidestream methane purge stream 646 could be combusted in the fired heater 470, but in methanol reactor and distillation unit 640, the methane purge stream 646 also contains useful amounts of CO and hydrogen that would otherwise be wasted. These can be sent as a secondary input to the separator subsystem 655.

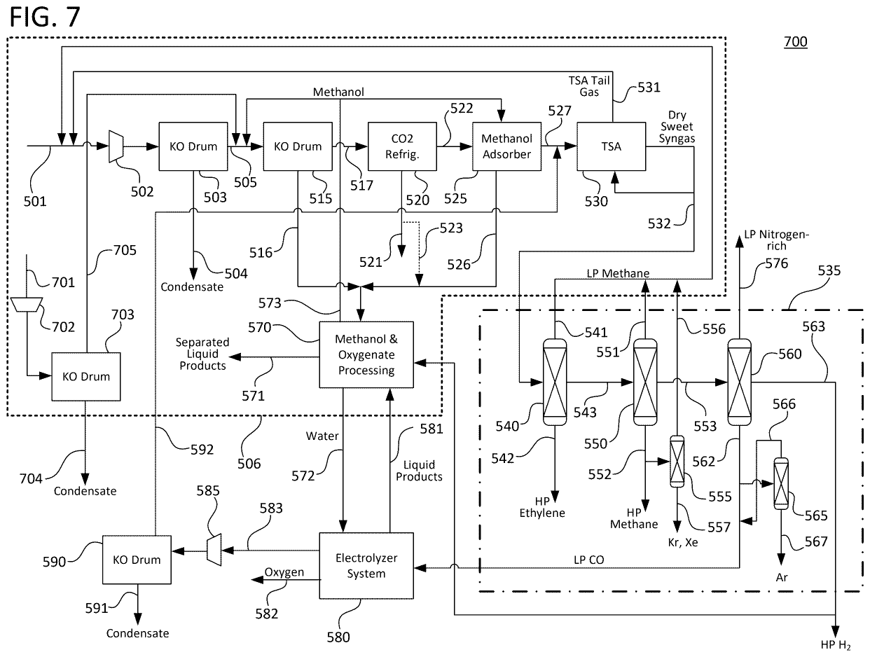

FIG. 7 illustrates an integrated separator/electrolyzer subsystem 700 according to specific embodiments of the invention. In this example, the separator/electrolyzer subsystem 700 shows a detailed view corresponding to the separator/electrolyzer subsystem 655 in FIG. 6. Operation of the separator/electrolyzer subsystem 700 proceeds similarly to subsystem 500 described in FIG. 5, and similarly to the discussion of FIG. 5, the process flow order of separator sections 506 and 535 and the electrolyzer system 580 can be shifted as desired. Most aspects are identical and will not be described separately, but separator/electrolyzer subsystem 700 allows an additional input with different composition than that of the PSA tail gas 654 listed as input tail gas stream 501 in FIG. 7. For example, methane purge stream 646 can also be input as feed stream 701. Feed stream 701 is compressed in a compressor 702 and then separated in a knockout (KO) drum 703. Most of the water in feed stream 701 is separated into a condensate 704. Stream 705 is then combined with stream 505, and then the rest of the separation/electrolysis is performed as described with respect to subsystem 500. In system 300, methanol produced in the optional methanol reactor as part of the separator and oxygenate processing subsystem 360 could be used just in the methanol drying portions or otherwise output as an additional product. In system 600, if a second methanol reactor is present, additional methanol produced in the reactor can be combined with methanol output stream 642 to increase the overall yield of the system 600.

In specific embodiments, excess hydrogen stream 363 that is not used in the internal methanol reactor can be output as hydrogen stream 367 that is combined with hydrogen recycle stream 352 for additional methanol production. In specific embodiments, separator/electrolyzer subsystem 700 may not have a separate methanol reactor but separated hydrogen and CO₂ within section 506 can be output to the methanol reactor and distillation unit 640 for additional production of methanol.

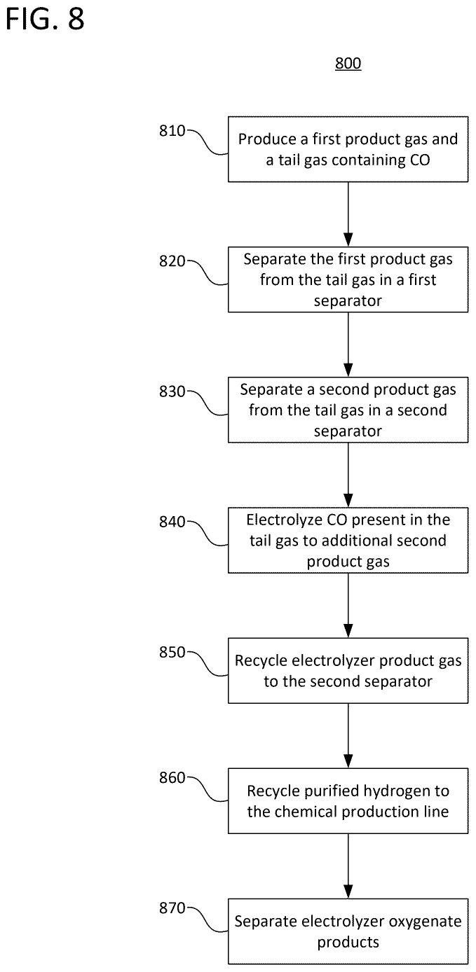

FIG. 8 shows a process 800 for producing and separating products in a chemical production line according to specific embodiments. In step 810, a first product gas is produced in a chemical production line. The first product gas may be mixed with a volume of carbon monoxide. In step 820, the first product gas is separated in a first separator from a tail gas that contains the volume of carbon monoxide. The tail gas can also have a number of other constituents, including the first product gas, hydrogen, carbon dioxide, water, and other inert gases such as nitrogen or noble gases. In step 830, the tail gas is fed into a second separator that has one or more separation stages, where a second product gas can be separated from the tail gas.

In step 840, at least part of the CO in the tail gas can be electrolyzed to secondary products such as a second product gas. Other liquid components such as oxygenates can be created at this time. In step 850, at least a portion of the electrolyzer product gas stream can be recycled to the second separator. Optionally, in step 860, purified hydrogen from the separation system can be sent back to the chemical production line. One example of this is sending hydrogen back to a methanol reactor to react with excess CO or CO₂. Also optionally, in step 870, another separator can be used to separate oxygenate products of the electrolyzer and additional products.

### EXAMPLE

Table 1 below provides an example material balance for PSA tail gas produced by a POx-based blue hydrogen system 300 as shown in FIG. 3, at various points through the separator/electrolyzer subsystem 355. Stream numbering is the same as in separator/electrolyzer subsystem 500 as shown in FIG. 5. The PSA tail gas composition may vary from the example. The CO concentration may be between 10-40 mol %, the CO₂ concentration may be between 5-35 mol %, the methane concentration may be between 3-10 mol %, the nitrogen concentration may be between 2-10 mol %, the argon concentration may be between 1-10 mol %, the krypton concentration may be between 0.0001-0.01 mol %, and the xenon concentration may be between 0.00001-0.001 mol %.

TABLE 1
Example Material Balance for POx Based Blue Hydrogen

| | | | | | | | | | Ethylene |
|---|---|---|---|---|---|---|---|---|---|
| | PSA Tail | | Refrig PSA | | Recycle | | | | Bottoms |
| | Gas Stream | | Tail Gas | | Hydrogen | | | | Product |
| | 501 | | Stream 522 | | Stream 563 | | Purge N₂ 576 | | 542 |
| Component | kgmol/h | mol % | kgmol/h | mol % | kgmol/h | mol % | kgmol/h | mol % | kgmol/h |
| H₂ | 956.21 | 39.80% | 951.42 | 43.70% | 651.94 | 95.24% | 6.34 | 11.09% | |
| CO | 887.01 | 36.90% | 878.14 | 40.33% | 13.04 | 1.90% | 6.34 | 11.09% | |
| CO₂ | 350.46 | 14.60% | 146.37 | 6.72% | | | | | |
| CH₄ | 145.11 | 6.00% | 137.85 | 6.33% | | | | | |
| N₂ | 64.03 | 2.70% | 63.39 | 2.91% | 19.56 | 2.86% | 44.47 | 77.82% | |
| Ar | 22.1 | 0.90% | 21.88 | 1.00% | | | | | |
| Kr | 0.02 | 0.00% | 0.02 | 0.00% | | | | | |
| Xe | 0 | 0.00% | 0 | 0.00% | | | | | |
| Ethylene | | | | | | | | | 347.28 |
| Acetic Acid | | | | | | | | | |
| Oxygen | | | | | | | | | |
| Total | 2,403 | 100% | 2,177 | 100.00% | 684.54 | 100.00% | 57.15 | 100.00% | 347.28 |

| | | | | Recycle | Argon | Xenon, |
|---|---|---|---|---|---|---|
| | Acetic | Oxygen | CO₂ to | Methane | Bottoms | Krypton |
| | Acid | Stream | Sequester | Stream | Product | Stream |
| | 571 | 582 | 521 | 552 | 567 | 557 |
| Component | kgmol/h | kgmol/h | kgmol/h | kgmol/h | kgmol/h | kgmol/h |
| H₂ | | | | | | |
| CO | | | | | | |
| CO₂ | | | 177.46 | | | |
| CH₄ | | | | 145.11 | | |
| N₂ | | | | | | |
| Ar | | | | | 22.1 | |
| Kr | | | | | | 0.024 |
| Xe | | | | | | 0.002 |
| Ethylene | | | | | | |
| Acetic Acid | 173.01 | | | | | |
| Oxygen | | 631.96 | | | | |
| Total | 173.01 | 631.96 | 177.46 | 145.11 | 22.1 | 0.03 |

While the specification has been described in detail with respect to specific embodiments of the invention, it will be appreciated that those skilled in the art, upon attaining an understanding of the foregoing, may readily conceive of alterations to, variations of, and equivalents to these embodiments. These and other modifications and variations to the present invention may be practiced by those skilled in the art, without departing from the scope of the present invention, which is more particularly set forth in the appended claims.

What is claimed is:

1. A chemical production system comprising:
a chemical production line with an output comprising a first product gas and carbon monoxide;
a first separator configured to separate the output into a volume of first product gas and a tail gas comprising a volume of carbon monoxide;
a second separator configured to separate a volume of a second product gas from the tail gas;
a carbon monoxide electrolyzer configured to receive at least a portion of the tail gas and convert at least a portion of the volume of carbon monoxide contained therein into a second volume of the second product gas; and
a recycle conduit configured to return gaseous products of the carbon monoxide electrolyzer to the second separator.

2. The chemical production system of claim 1, wherein the second separator comprises at least one cryogenic separation unit.

3. The chemical production system of claim 2, wherein the at least one cryogenic separation unit operates at multiple pressure stages with sequential condensation and stripping of ethylene, methane, and carbon monoxide.

4. The chemical production system of claim 2, wherein the at least one cryogenic separation unit comprises one or more cryogenic separation stages.

5. The chemical production system of claim 4, wherein a product of the one or more cryogenic separation stages is the second product gas.

6. The chemical production system of claim 5, wherein the second product gas is ethylene.

7. The chemical production system of claim 4, wherein a product of the one or more cryogenic separation stages is a nitrogen purge stream having greater than 60 mol % nitrogen.

8. The chemical production system of claim 2, further comprising a temperature swing adsorption unit upstream of the at least one cryogenic separation unit configured to remove at least one of residual water, methanol, and oxygenates.

9. The chemical production system of claim 2, further comprising one or more sidestream cryogenic separators configured to separate one or more of argon, krypton, and xenon.

10. The chemical production system of claim 1, wherein the carbon monoxide electrolyzer also produces a volume of hydrogen, and the volume of hydrogen is fed to a stage in the chemical production line.

11. The chemical production system of claim 1, wherein the carbon monoxide electrolyzer also produces a volume of oxygen, and the volume of oxygen is fed to a stage in the chemical production line.

12. The chemical production system of claim 11, wherein the stage in the chemical production line is an autothermal reformer or a partial oxidation reactor.

13. The chemical production system of claim 1, wherein the gaseous products of the carbon monoxide electrolyzer further comprise hydrogen.

14. The chemical production system of claim 1, wherein the second separator produces a high-purity hydrogen stream from hydrogen contained in at least one of the tail gas and the gaseous products of the carbon monoxide electrolyzer.

15. The chemical production system of claim 14, wherein the second separator further comprises a methanol production reactor.

16. The chemical production system of claim 15, wherein methanol produced in the methanol production reactor valorizes carbon dioxide from the chemical production line with a volume of hydrogen from the high-purity hydrogen stream.

17. The chemical production system of claim 15, wherein methanol produced in the methanol production reactor is used as an absorbent to remove carbon dioxide from the tail gas.

18. The chemical production system of claim 14, wherein at least a portion of the high-purity hydrogen stream is fed to a stage in the chemical production line.

19. The chemical production system of claim 1, wherein the portion of the tail gas received by the carbon monoxide electrolyzer contains between 50-80 mol % carbon monoxide.

20. The chemical production system of claim 1, wherein the second separator further comprises one or more additional stages including a temperature swing adsorber, a knockout drum, a liquid drying agent, and a refrigeration system.

21. The chemical production system of claim 1, wherein the chemical production line is a methanol production line.

22. The chemical production system of claim 1, wherein the chemical production line is a blue hydrogen production line.

23. The chemical production system of claim 1, wherein the carbon monoxide electrolyzer is configured to convert at least a portion of the carbon monoxide into one or more liquid oxygenate products.

24. The chemical production system of claim 1, wherein the chemical production line comprises a natural gas reforming reactor.

25. The chemical production system of claim 24, wherein the natural gas reforming reactor is an autothermal reformer, a partial-oxidation reactor, or a steam-methane reformer.

26. A method of operating a chemical production line, comprising:
producing a first product gas and a tail gas containing a volume of carbon monoxide;
separating a first volume of the first product gas from the tail gas containing the volume of carbon monoxide in a first separator;
separating a first volume of a second product gas from the tail gas in a second separator;
electrolyzing, in a carbon monoxide electrolyzer, at least a portion of the tail gas to convert at least a portion of the volume of carbon monoxide into an electrolyzer product gas having a second volume of the second product gas; and
recycling at least a portion of the electrolyzer product gas to the second separator.

27. The method of claim 26, wherein the electrolyzing, in the carbon monoxide electrolyzer, at least a portion of the tail gas also produces an additional product comprising an oxygenate.

28. The method of claim 26, further comprising recycling purified hydrogen from the second separator to a reactor in the chemical production line.

29. A blue hydrogen production system comprising:
a reforming reactor, selected from an autothermal reformer, a partial-oxidation reactor, and a steam-methane reformer, that produces a reforming reactor output comprising a first volume of carbon monoxide and a first volume of hydrogen;
a catalytic water-gas-shift reactor configured to produce, from the reforming reactor output, a shifted output stream comprising a second volume of carbon monoxide, a second volume of hydrogen, and a volume of carbon dioxide;
a first separator configured to separate the shifted output stream into a volume of hydrogen and a tail gas stream comprising the second volume of carbon monoxide, the first separator selected from a pressure swing adsorber, a membrane separator, and a cryogenic separator;
a carbon-monoxide electrolyzer configured to receive at least a portion of the tail gas stream and to produce an electrolyzer product gas stream comprising a second product gas from at least a portion of the second volume of carbon monoxide; and
a second separator comprising a cryogenic separation system, the cryogenic separation system configured to separate each of the tail gas stream and the electrolyzer product gas stream into one or more of (i) a hydrogen stream, (ii) a carbon-monoxide stream, (iii) a nitrogen-rich purge stream, and (iv) a stream containing a volume of the second product gas.

30. The blue hydrogen production system of claim 29, further comprising an additional methanol synthesis subloop, wherein the tail gas stream further comprises a volume of carbon dioxide and the additional methanol synthesis subloop is configured to combine the volume of carbon dioxide with a portion of the hydrogen stream from the second separator to produce additional methanol.

31. A methanol production system comprising:
a reforming reactor, selected from an autothermal reformer, a partial-oxidation reactor, and a steam-methane reformer, that receives a hydrocarbon as an input and produces a reforming reactor output comprising carbon monoxide, carbon dioxide, and hydrogen;
a methanol synthesis reactor configured to produce, from the reforming reactor output, an output stream comprising methanol and residual carbon monoxide;
a first separator configured to separate the output stream into a volume of methanol and a tail gas stream comprising a volume of the residual carbon monoxide, the first separator selected from a pressure swing adsorber, a membrane separator, and a cryogenic separator;
a carbon monoxide electrolyzer configured to receive at least a portion of the tail gas stream and to produce an electrolyzer product gas stream comprising a second product gas from at least a portion of the volume of the residual carbon monoxide; and
a second separator comprising a cryogenic separation system, the cryogenic separation system configured to separate each of the tail gas stream and the electrolyzer product gas stream into one or more of (i) a hydrogen stream, (ii) a carbon-monoxide stream, (iii) a nitrogen-rich purge stream, and (iv) a stream containing a volume of the second product gas.

32. The methanol production system of claim 31, further comprising an additional methanol synthesis subloop, wherein the tail gas stream further comprises a volume of carbon dioxide, and the additional methanol synthesis subloop is configured to combine the volume of carbon dioxide with a portion of the hydrogen stream from the second separator to produce additional methanol.
